(19) United States
(12) Patent Application Publication
(10) Pub. No.: US 2016/0023978 A1
(43) Pub. Date: Jan. 28, 2016

(54) 6-HYDROXY-2-NAPHTHALENYL FLUORENE DERIVATIVES AND LENS AND CAMERA MODULE USING THE SAME

(71) Applicant: SAMSUNG ELECTRO-MECHANICS CO., LTD., Suwon-Si (KR)

(72) Inventors: Hoe Chul JUNG, Seoul (KR); Jun Young KIM, Seoul (KR); In Cheol CHANG, Sungnam-si (KR); Ichiro OGURA, Suwon-si (KR)

(73) Assignee: SAMSUNG ELECTRO-MECHANICS CO., LTD., Suwon-Si (KR)

(21) Appl. No.: 14/807,269

(22) Filed: Jul. 23, 2015

(30) Foreign Application Priority Data
Jul. 28, 2014 (KR) 10-2014-0095904
Oct. 13, 2014 (KR) 10-2014-0137440

(51) Int. Cl. C07C 39/14; C08G 61/10; G02B 1/04; C07C 43/23

(52) U.S. Cl. CPC C07C 39/14; C07C 43/23; C08G 61/10; G02B 1/041

(57) ABSTRACT

Disclosed herein are 6-hydroxy-2-naphthalenyl fluorene derivatives and a lens and a camera module using the same.

### CROSS REFERENCE(S) TO RELATED APPLICATIONS

This application claims the benefit under 35 U.S.C. Section [120, 119, 119(e)] of Korean Patent Application Serial Nos. 10-2014-0095904 and 10-2014-0137440, entitled “6-Hydroxy-2-naphthalenyl Fluorene Derivatives and Lens and Camera Module Using the Same” filed on Jul. 28, 2014 and Oct. 13, 2014, which are hereby incorporated by reference in their entirety into this application.

### BACKGROUND OF THE INVENTION

1. Technical Field

The present disclosure relates to 6-hydroxy-2-naphthalenyl fluorene derivatives and a lens and a camera module using the same.

2. Description of the Related Art

An optical glass or optical transparent resin has been used as a material of an optical element used in optical systems of various cameras such as a camera for a smart phone, a video camera, and the like.

There are various kinds of optical glasses having excellent heat resistance, transparency, dimensional stability, chemical resistance, or the like, and having various refractive indexes (nD) or Abbe's numbers (uD), but there are problems such as an expensive material, poor formability, and low productivity. Particularly, since a significantly high technology and a high cost are required in order to process the optical glass into an aspheric lens used in aberration correction, there are various limitations in actually using the optical glass.

Meanwhile, an optical lens made of an optical transparent resin, particularly, a thermoplastic transparent resin may be mass-produced by molding injection and easily manufactured in an aspheric lens form, such that the optical lens has been used as a camera lens.

A resin composition having a fluorene compound as a monomer unit and containing a polycondensation-polyaddition polymer having at least one sulfur atom in a repeating unit, and an optical element formed by injection molding the resin composition have been disclosed in Patent Document 1.

### SUMMARY OF THE INVENTION

An object of the present disclosure is to provide 6-hydroxy-2-naphthalenyl fluorene derivatives having high polarity and a low molecular volume so that excellent optical properties are exhibited.

Another object of the present disclosure is to provide 6-hydroxy-2-naphthalenyl fluorene derivatives having a low glass transition temperature (Tg) so as to be easily worked and molded.

Another object of the present disclosure is to provide 6-hydroxy-2-naphthalenyl fluorene derivatives having high transparency.

Another object of the present disclosure is to provide eco-friendly 6-hydroxy-2-naphthalenyl fluorene derivatives.

Another object of the present disclosure is to provide a copolymer of the 6-hydroxy-2-naphthalenyl fluorene derivative.

Another object of the present disclosure is to provide a lens formed by molding a copolymer of the 6-hydroxy-2-naphthalenyl fluorene derivative.

Another object of the present disclosure is to provide a camera module manufactured using a lens formed by molding a copolymer of the 6-hydroxy-2-naphthalenyl fluorene derivative.

According to an exemplary embodiment of the present disclosure, there is provided 6-hydroxy-2-naphthalenyl fluorene derivatives represented by the following Chemical Formula 1.

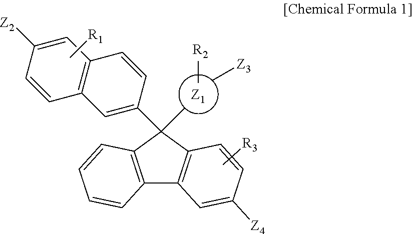

In Chemical Formula 1, R₁, R₂, and R₃ may be the same or different and each be hydrogen (H) or represented by the following Chemical Formula 2, at least one of R₁, R₂, and R₃ being selected from compounds represented by the following Chemical Formula 2. A substitution position of R₁ in a naphthyl group is not particularly limited. Further, Z₁ may be H or be selected from a phenyl group, a naphthyl group, a biphenyl group, an anthryl group, and a phenanthryl group which have a C1-C4 alkyl substituent group.

Z₂, Z₃, and Z₄ are the same or different and are each represented by an —H, —O—H, or —OCH₂CH₂O—H group, at least one of Z₂, Z₃, and Z₄ being —H; and A substitution position of Z₄ in a fluorene benzene ring of Chemical Formula 1 is not particularly limited.

—R₄—Ar— [Chemical Formula 2]

In Chemical Formula 2, R₄ is a C1-C5 alkyl group and Ar is a C6-C22 aryl group.

Ar may be H or be selected from a phenyl group, a naphthyl group, a biphenyl group, an anthryl group, and a phenanthryl group which have a C1-C4 alkyl substituent group.

According to another exemplary embodiment of the present disclosure, there is provided a copolymer of a compound represented by Chemical Formula 1.

According to another exemplary embodiment of the present disclosure, there is provided a lens manufactured by molding a copolymer of a compound represented by Chemical Formula 1.

Various features and advantages of the present disclosure will be more obvious from the following description with reference to the accompanying drawings.

The terms and words used in the present specification and claims should not be interpreted as being limited to typical meanings or dictionary definitions, but should be interpreted as having meanings and concepts relevant to the technical scope of the present disclosure based on the rule according to which an inventor can appropriately define the concept of the term to describe most appropriately the best method he or she knows for carrying out the disclosure.

### BRIEF DESCRIPTION OF THE DRAWINGS

FIG. 1 is a view showing a schematic synthetic scheme for a copolymer of a 6-hydroxy-2-naphthalenyl fluorene derivative according to an exemplary embodiment of the present disclosure.

### DESCRIPTION OF THE PREFERRED EMBODIMENTS

The above and other objects, features and advantages of the present disclosure will be more clearly understood from preferred embodiments and the following detailed description taken in conjunction with the accompanying drawings. In the specification, in adding reference numerals to components throughout the drawings, it is to be noted that like reference numerals designate like components even though components are shown in different drawings. Further, when it is determined that the detailed description of the known art related to the present disclosure may obscure the gist of the present disclosure, the detailed description thereof will be omitted. In the description, the terms “first”, “second”, and so on, are used to distinguish one element from another element, and the elements are not defined by the above terms.

Hereinafter, exemplary embodiments of the present disclosure will be described in detail with reference to the accompanying drawings.

### 6-Hydroxy-2-naphthalenyl fluorene derivatives

The 6-hydroxy-2-naphthalenyl fluorene derivatives according to an exemplary embodiment of the present disclosure is represented by the following Chemical Formula 1:

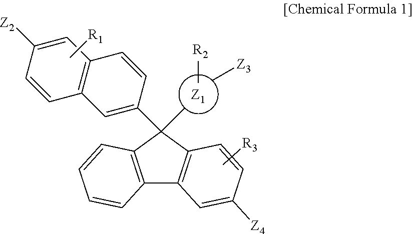

In Chemical Formula 1, R₁, R₂, and R₃ may be the same or different and each be hydrogen (H) or represented by the following Chemical Formula 2, at least one of R₁, R₂, and R₃ being selected from compounds represented by the following Chemical Formula 2. A substitution position of R₁ in a naphthyl group is not particularly limited. Further, Z₁ may be H or be selected from a phenyl group, a naphthyl group, a biphenyl group, an anthryl group, and a phenanthryl group which have a C1-C4 alkyl substituent group. Z₂, Z₃, and Z₄ may be the same or different and each be represented by an —H, —O—H, or —OCH₂CH₂O—H group, at least one of Z₂, Z₃, and Z₄ being —H. A substitution position of Z₄ in a fluorene benzene ring of Chemical Formula 1 is not particularly limited.

—R₄—Ar— [Chemical Formula 2]

In Chemical Formula 2, R₄ is a C1-C5 alkyl group and Ar is a C6-C22 aryl group.

The Ar may be selected from the group consisting of a phenyl group, a naphthyl group, a biphenyl group, an anthryl group, and a phenanthryl group.

In the 6-hydroxy-2-naphthalenyl fluorene derivative, an aryl group having a low molecular volume is introduced in at least one aryl moiety of a 6-hydroxy-2-naphthalenyl fluorene moiety, a polarity of the 6-hydroxy-2-naphthalenyl fluorene derivative itself may be increased, thereby making it possible to improve a refractive index.

Further, an alkyl group having a rotatable bond is disposed at the ortho position of the aryl group, such that a glass transition temperature (Tg) may be decreased. As a result, a viscosity may be decreased, such that moldability and workability may be improved.

A halogen substituent such as bromine (Br) or chlorine (Cl), that is generally used for improving optical properties but causing a dioxine problem is not introduced in the 6-hydroxy-2-naphthalenyl fluorene moiety, such that the 6-hydroxy-2-naphthalenyl fluorene derivative is eco-friendly, and a sulfur (S) or nitrogen (N) atom is not used, such that transparency may be secured.

The 6-hydroxy-2-naphthalenyl fluorene derivative represented by Chemical Formula 1 may be a compound represented by the following Chemical Formula 3.

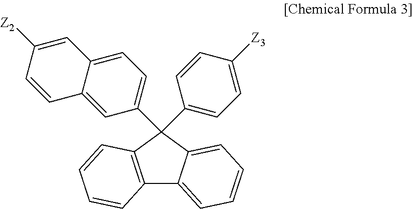

Here, Z₂ and Z₃ may be the same or different and be represented by an —O—H or —OCH₂CH₂O—H group.

The 6-hydroxy-2-naphthalenyl fluorene derivative represented by Chemical Formula 1 may be a compound represented by the following Chemical Formula 4.

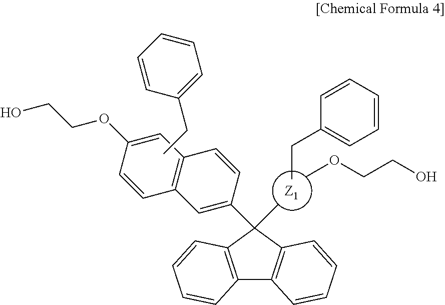

More specifically, in the compound represented by Chemical Formula 4, a benzyl group is introduced in two aryl moieties of the 6-hydroxy-2-naphthalenyl fluorene moiety, such that the polarity of the 6-hydroxy-2-naphthalenyl fluorene derivative itself may be increased, thereby making it possible to improve the refractive index. In addition, a methyl group is disposed at the ortho position of the benzyl group, such that the substituent itself may rotate, and accordingly, the viscosity of the derivative may be decreased, thereby making it possible to improve moldability and workability. Here, Z₁ may be H or be selected from a phenyl group, a naphthyl group, a biphenyl group, an anthryl group, and a phenanthryl group which have a C1-C4 alkyl substituent group. In this case, a substitution position of the benzyl group in the naphthyl group is not particularly limited.

The 6-hydroxy-2-naphthalenyl fluorene derivative represented by Chemical Formula 1 may be a compound represented by the following Chemical Formula 5.

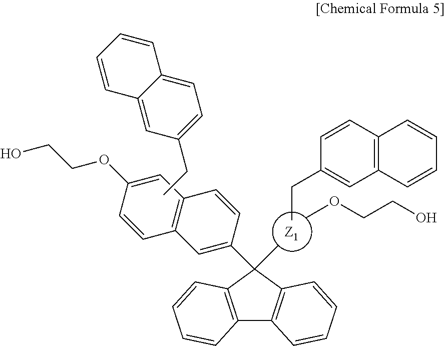

More specifically, in the compound represented by Chemical Formula 5, a methyl naphthyl group is introduced in two aryl moieties of the 6-hydroxy-2-naphthalenyl fluorene moiety, such that the polarity of the 6-hydroxy-2-naphthalenyl fluorene derivative itself may be increased, thereby making it possible to improve a refractive index. In addition, a methyl group is disposed at the ortho position of the methyl naphthyl group, such that the substituent itself may rotate, and accordingly, the viscosity of the derivative may be decreased, thereby making it possible to improve moldability and workability. Here, Z₁ may be H or be selected from a phenyl group, a naphthyl group, a biphenyl group, an anthryl group, and a phenanthryl group which have a C1-C4 alkyl substituent group. In this case, a substitution position of the methyl naphthyl group in the naphthyl group is not particularly limited.

The 6-hydroxy-2-naphthalenyl fluorene derivative represented by Chemical Formula 1 may be a compound represented by the following Chemical Formula 6.

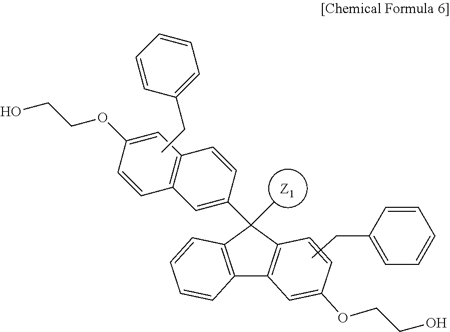

More specifically, in the compound represented by Chemical Formula 6, a benzyl group is introduced in two aryl moieties of the 6-hydroxy-2-naphthalenyl fluorene moiety, such that the polarity of the 6-hydroxy-2-naphthalenyl fluorene derivative itself may be increased, thereby making it possible to improve a refractive index. In addition, a methyl group is disposed at the ortho position of the benzyl group, such that the substituent itself may rotate, and accordingly, the viscosity of the derivative may be decreased, thereby making it possible to improve moldability and workability. Here, Z₁ may be H or be selected from a phenyl group, a naphthyl group, a biphenyl group, an anthryl group, and a phenanthryl group which have a C1-C4 alkyl substituent group. In this case, a substitution position of the benzyl group in the naphthyl group is not particularly limited. In addition, a substitution position of an —OCH₂CH₂O—H group of Chemical Formula 6 substituted at a Z₄ position of Chemical Formula 1 in the fluorene benzene ring is not particularly limited.

The 6-hydroxy-2-naphthalenyl fluorene derivative represented by Chemical Formula 1 may be a compound represented by the following Chemical Formula 7.

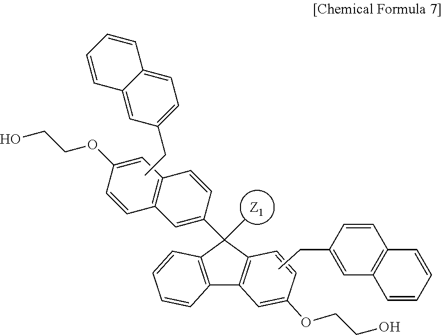

More specifically, in the compound represented by Chemical Formula 7, a methyl naphthyl group is introduced in two aryl moieties of the 6-hydroxy-2-naphthalenyl fluorene moiety, such that the polarity of the 6-hydroxy-2-naphthalenyl fluorene derivative itself may be increased, thereby making it possible to improve a refractive index. In addition, a methyl group is disposed at the ortho position of the methyl naphthyl group, such that the substituent itself may rotate, and accordingly, the viscosity of the derivative may be decreased, thereby making it possible to improve moldability and workability. Here, Z₁ may be H or be selected from a phenyl group, a naphthyl group, a biphenyl group, an anthryl group, and a phenanthryl group which have a C1-C4 alkyl substituent group. In this case, a substitution position of the methyl naphthyl group in the naphthyl group is not particularly limited. In addition, a substitution position of an —OCH₂CH₂O—H group of Chemical Formula 7 substituted at a Z₄ position of Chemical Formula 1 in the fluorene benzene ring is not particularly limited.

### Preparation Method of 6-Hydroxy-2-naphthalenyl fluorene derivatives

Hereinafter, a preparation method of 6-hydroxy-2-naphthalenyl fluorene derivatives according to the exemplary embodiment of the present disclosure will be described in detail. However, specific Reaction Formulas described below is to describe the preparation method by way of example, and it may be appreciated by those skilled in the art that the preparation method of 6-hydroxy-2-naphthalenyl fluorene derivatives is not limited thereto.

As an example, the compound represented by Chemical Formula 3 may be synthesized using 2-bromo-6-methoxynaphthalene and 9-fluorenone as starting materials as shown in the following Reaction Formula 1.

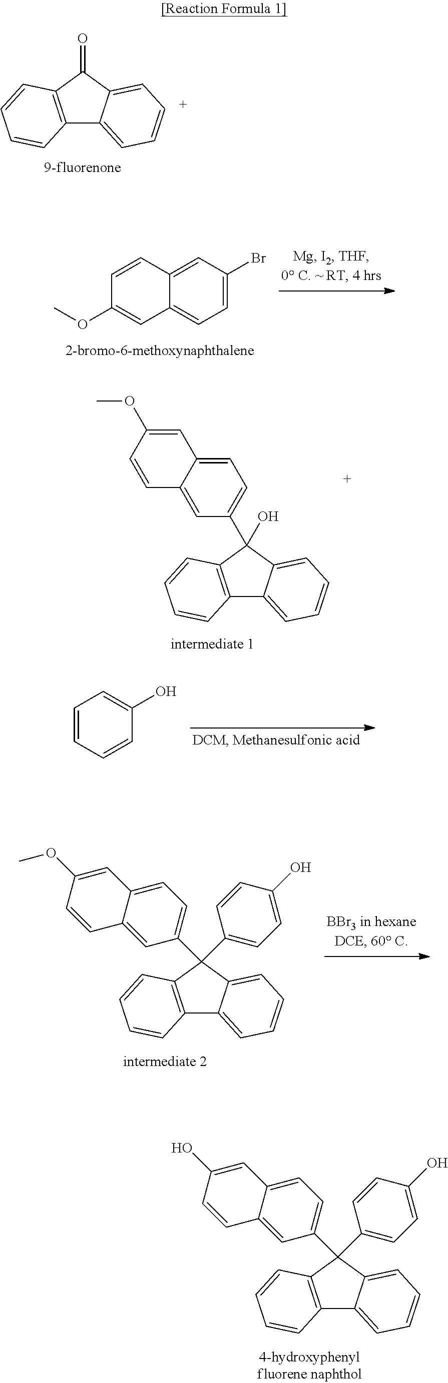 [SMILES: O=C1c2ccccc2-c2ccccc21 ; COc1ccc2cc(Br)ccc2c1 ; COc1ccc2cc(C3(O)c4ccccc4-c4ccccc43)ccc2c1 ; Oc1ccccc1 ; COc1ccc2cc(C3(c4ccc(O)cc4)c4ccccc4-c4ccccc43)ccc2c1 ; Oc1ccc(C2(c3ccc4cc(O)ccc4c3)c3ccccc3-c3ccccc32)cc1]

As an example, the compound represented by Chemical Formula 4 may be synthesized using naphthalene-2-ol and 9-fluorenone as starting materials as shown in the following Reaction Formula 2.

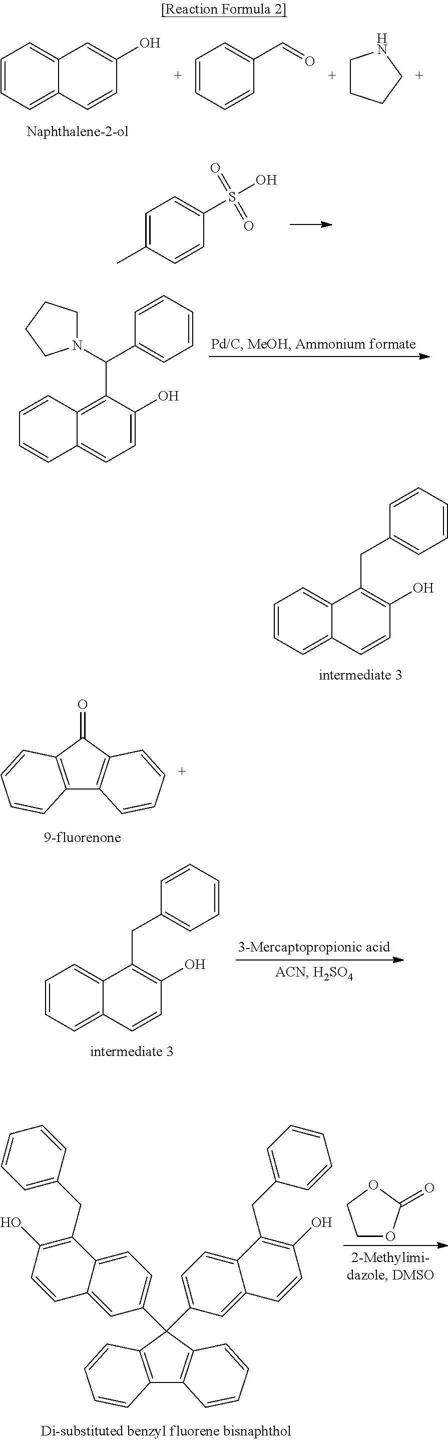

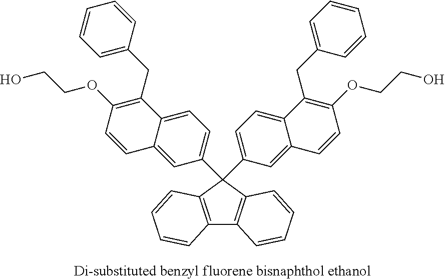 [SMILES: OCCOc1ccc2cc(C3(c4ccc5c(Cc6ccccc6)c(OCCO)ccc5c4)c4ccccc4-c4ccccc43)ccc2c1Cc1ccccc1]

As another example, the compound represented by Chemical Formula 4 may be synthesized through a reaction as shown in the following Reaction Formula 3.

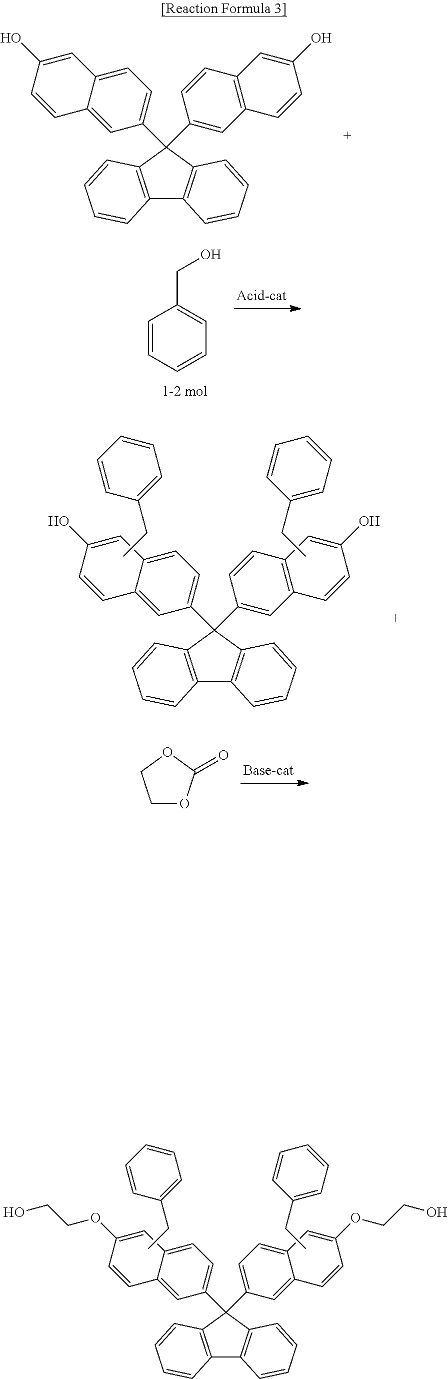

Here, a base-catalyst may be used instead of an acid-catalyst, and benzyl chloride may be used instead of benzyl alcohol reacting with 6-hydroxy-2-naphthalenyl fluorene bisnaphthol.

As an example, the compound represented by Chemical Formula 5 may be synthesized using 6-hydroxy-2-naphthalenyl fluorene bisnaphthol as a starting material as shown in the following Reaction Formula 4.

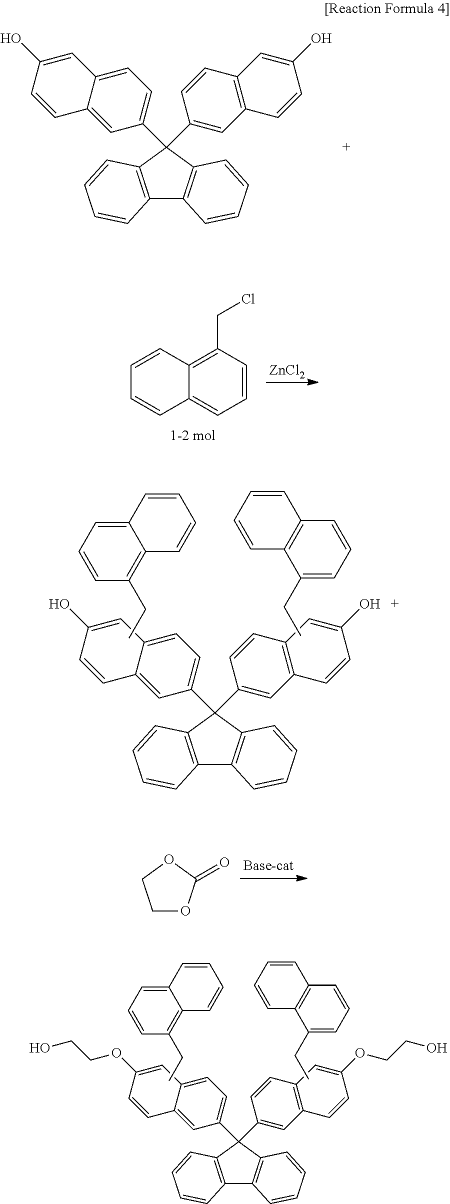

Here, an acid-catalyst may be used instead of the base-catalyst, and 1-naphthyl methanol may be used instead of 1-(chloromethyl) naphthalene reacting with 6-hydroxy-2-naphthalenyl fluorene bisnaphthol.

As an example, the compound represented by Chemical Formula 6 may be synthesized through a reaction as shown in the following Reaction Formula 5.

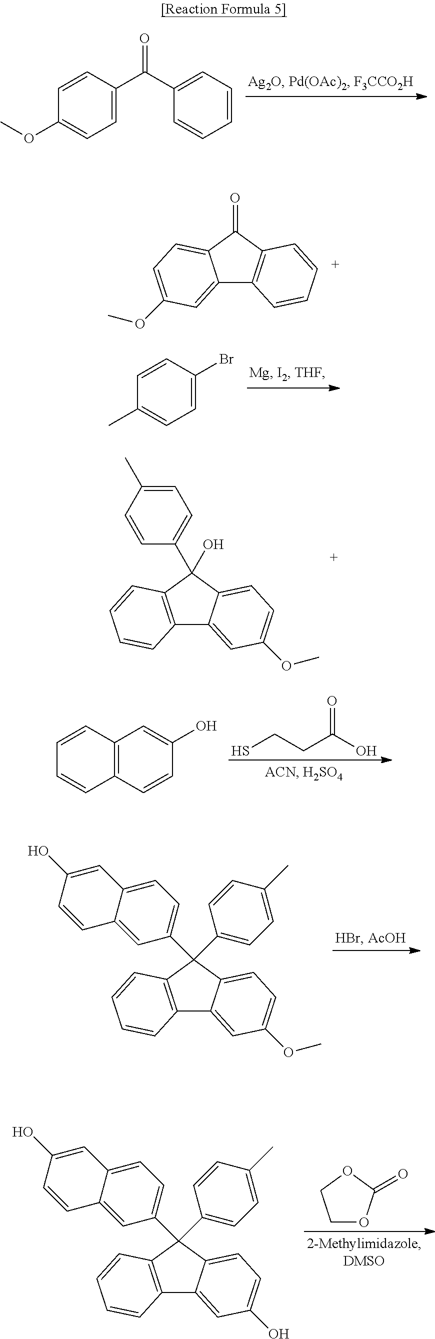

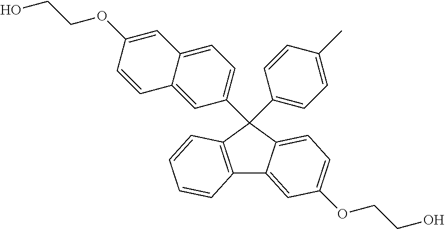 [SMILES: Cc1ccc(C2(c3ccc4cc(OCCO)ccc4c3)c3ccccc3-c3cc(OCCO)ccc32)cc1]

As another example, the compound represented by Chemical Formula 6 may be synthesized through a reaction as shown in the following Reaction Formula 6.

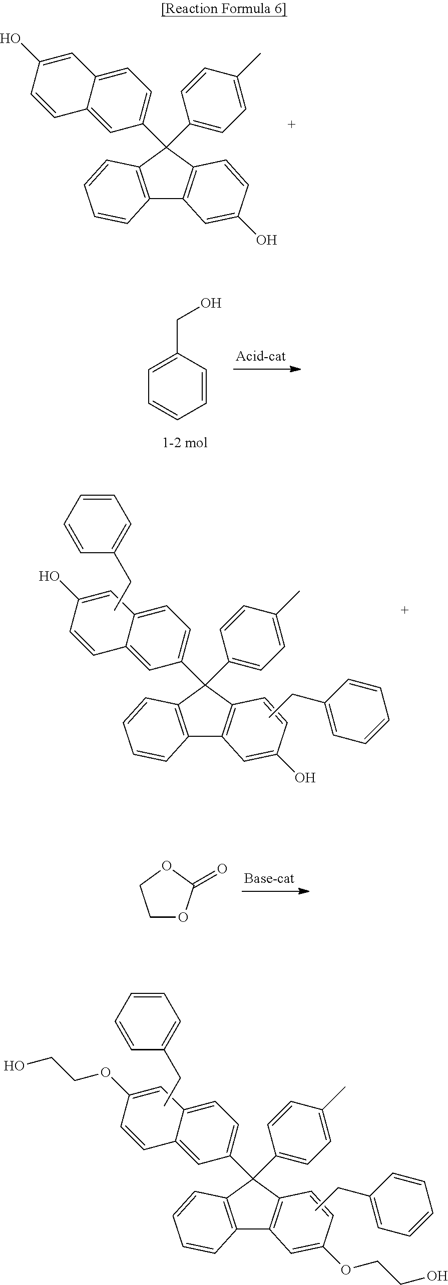

Here, a base-catalyst may be used instead of an acid-catalyst, and benzyl chloride may be used instead of benzyl alcohol reacting with 6-hydroxy-2-naphthalenyl fluorene derivative.

As an example, the compound represented by Chemical Formula 7 may be synthesized through a reaction as shown in the following Reaction Formula 7.

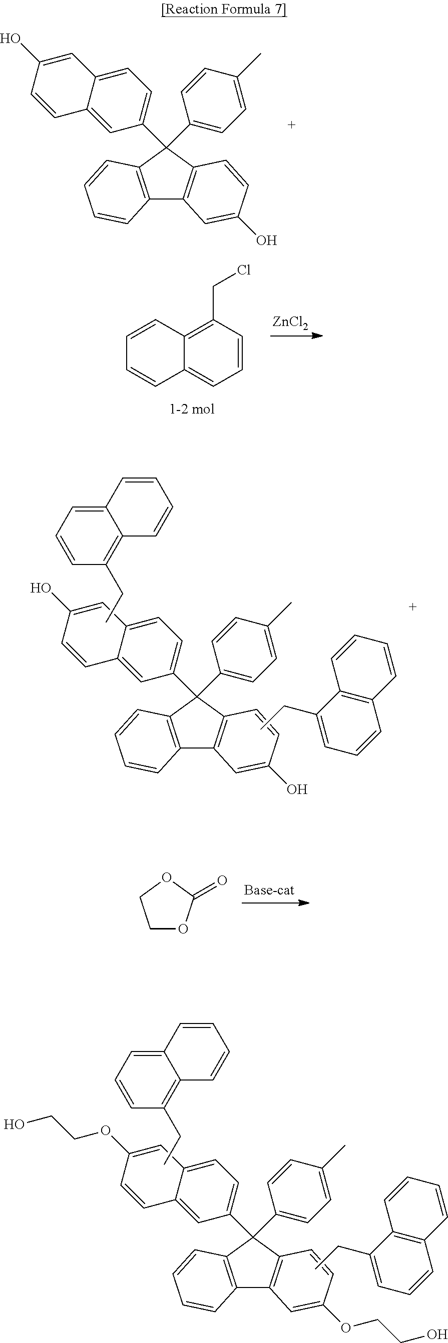

Here, an acid-catalyst may be used instead of a base-catalyst, and 1-naphthyl methanol may be used instead of 1-(chloromethyl) naphthalene reacting with 6-hydroxy-2-naphthalenyl fluorene derivative.

Copolymer

A copolymer according to an exemplary embodiment of the present disclosure is a copolymer of a 6-hydroxy-2-naphthalenyl fluorene derivative represented by Chemical Formula 1. Hereinafter, specific examples of the copolymer will be described, but the following compounds are provided for illustrative purpose, and the copolymer according to the present disclosure is not limited thereto.

For example, in the case in which the copolymer is obtained by copolymerizing the 6-hydroxy-2-naphthalenyl fluorene derivate of Chemical Formula 3 in which the benzyl group is introduced in two aryl moieties of the 6-hydroxy-2-naphthylenyl fluorene moiety, the copolymer may be represented by the following Chemical Formula 8.

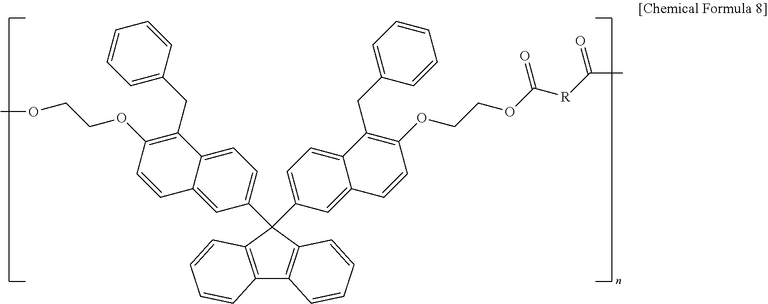

In Chemical Formula 8, R, which is a dicarboxylic acid component, may be a dicarboxylic acid, a dicarboxylic acid derivative (dicarboxylic acid derivative capable of forming an ester bond, ester-forming dicarboxylic acid derivative), or the like. One kind of dicarboxylic acid component may be used alone, or a combination of two or more dicarboxylic acid components may be used. For example, the dicarboxylic acid and the derivative thereof may be used as the dicarboxylic acid component. Representative examples of the dicarboxylic acid may include aliphatic dicarboxylic acids such as alkane dicarboxylic acids (oxalic acid, malonic acid, succinic acid, glutaric acid, adipic acid, and the like), alkene dicarboxylic acids (maleic acid, fumaric acid, and the like); alicyclic dicarboxylic acids such as cycloalkane dicarboxylic acids (cyclohexane dicarboxylic acid, and the like), di- or tri-cycloalkane dicarboxylic acids (decalin dicarboxylic acid, norbornane dicarboxylic acid, adamantane dicarboxylic acid, and the like), and the like; aromatic dicarboxylic acids such as arene dicarboxylic acids (terephthalic acid, isophthalic acid, phthalic acid, 2,6-naphthalene dicarboxylic acid, 1,8-naphthalene dicarboxylic acid, anthracene dicarboxylic acid, and the like), biphenyl dicarboxylic acids (2,2′-biphenyl dicarboxylic acid, and the like), and the like. In addition, reactive derivative thereof (derivatives capable of forming esters, for example, acid hydrides such as hexahydrophthalic anhydride, tetrahydro phthalic anhydride, and the like, lower (C1-C4) alkyl esters such as dimethyl ester, diethylester, and the like, acid halides corresponding to the dicarboxylic acids, and the like) may be used. R may be changed depending on the kind of monomer actually used at the time of copolymerization.

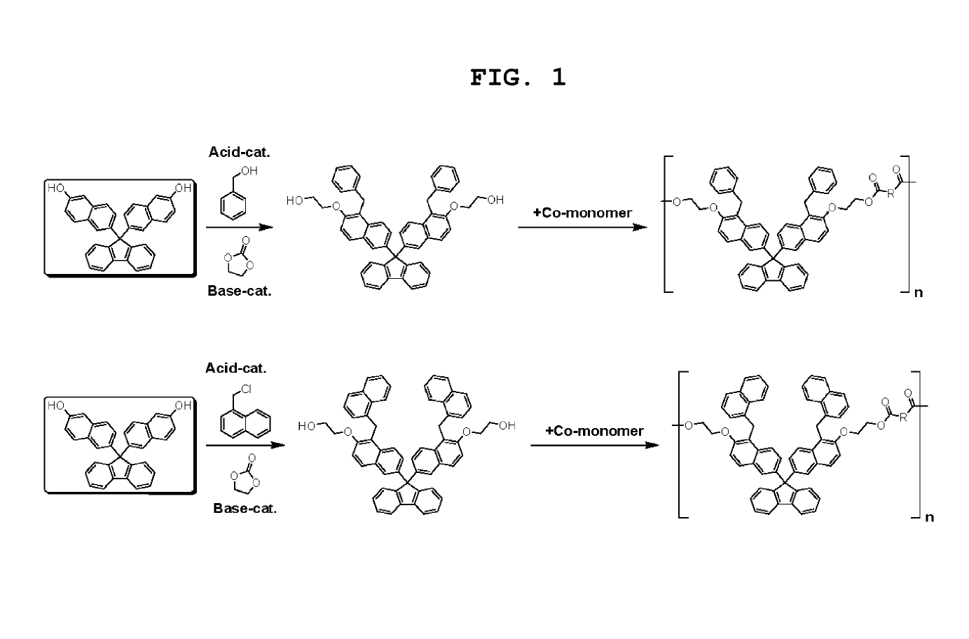

FIG. 1 is a view showing a Reaction Formula indicating a schematic synthesis scheme for the copolymer represented by Reaction Formula 3[u1].

Referring to FIG. 1, the polymer may be obtained through a carbonation reaction by mixing a 6-hydroxy-2-naphthalenyl fluorene derivative and a monomer used at the time of copolymerization in the presence of a mixed solution of an aqueous base solution and an organic solvent.

A preparation method of the copolymer is not particularly limited, but a general copolymerization method known in the art may be applied.

Lens and Camera Module

A lens according to an exemplary embodiment of the present disclosure is obtained by molding a copolymer of a 6-hydroxy-2-naphthalenyl fluorene derivative represented by Chemical Formula 1.

The 6-hydroxy-2-naphthalenyl fluorene derivative and the copolymer thereof are the same as those as described above, and a detailed description thereof will be omitted.

The lens obtained by molding the copolymer of the 6-hydroxy-2-naphthalenyl fluorene derivative has high refractive properties (refractive index of 1.60 or more) and overall optical properties thereof such as transparency, and the like, are excellent.

The lens according to an exemplary embodiment of the present disclosure may be obtained, for example, by injection molding the copolymer of the 6-hydroxy-2-naphthalenyl fluorene derivative in a lens shape using an injection molding machine or injection press molding machine.

If necessary, the lens may be used in an aspheric lens shape. Among optical lenses, an aspheric lens is useful as a camera lens. If necessary, a coat layer such as an anti-reflection layer or a hard coat layer, may be formed on a surface of the lens.

The lens may be used as various lenses such as pickup lenses, f−θ lens, eyeglass lenses, and the like. More specifically, the lens may be used as a lens of a single lens reflex camera, a digital steel camera, a video camera, a camera mounted mobile phone, a lens mounted film, a telescope, a binocular telescope, microscope, a projector, or the like. Further, a camera module using the lens may be manufactured and used.

Hereinafter, the present disclosure will be described with reference to Examples, but the present disclosure is not limited thereto.

### Synthesis of 9-(6-Methoxynaphthalene-2-yl)-9H-fluorene-9-ol (Intermediate 1)

2.37 g of 2-bromo-6-methoxynaphthalene (10 mmol) was put into a 100 mL round bottom flask with a magnetic stirring bar and dissolved in 10 mL anhydrous tetrahydrofuran (THF). After 480 mg of magnesium and a small amount of iodine were added thereto and stirred under nitrogen atmosphere, it was confirmed that a color of a solution was changed into grey color. Thereafter, the mixture was cooled to 0 degrees in an ice bath. 901 mg of 9-fluorenone (5 mmol) dissolved using 5 mL of anhydrous THF was slowly added dropwise thereto under nitrogen atmosphere. A temperature was raised to room temperature and the stirring was maintained for 4 hours. After a reaction end point was confirmed using thin layer chromatography (TLC) and the resultant was cooled in an ice bath, the reaction was terminated by 30 mL of saturated aqueous ammonium chloride solution. After the resultant was extracted with dichloromethane, and an organic layer was washed with Brine, dried over magnesium sulfate, filtered, and concentrated, thereby obtaining a crude product as an oil. The crude product as an oil was purified using a silica gel column (developing solution: 5% to 20% ethyl acetate/hexane), thereby obtaining 1.68 g of yellow oil. Thereafter, it was confirmed using nuclear magnetic resonance (NMR) that the obtained oil was 9-(6-methoxynaphthalene-2-yl)-9H-fluorene-9-ol. Yield: 99%, ¹H NMR (700 MHz, CDCl₃) δ8.09 (d, J=1.4 Hz, 1H), 7.74 (d, J=9.0 Hz, 1H), 7.70 (d, J=7.6 Hz, 2H), 7.56 (d, J=8.7 Hz, 1H), 7.38 (td, J=7.5, 1.0 Hz, 2H), 7.35 (d, J=7.5 Hz, 2H), 7.26-7.23 (m, 2H), 7.13 (dt, J=8.6, 2.4 Hz, 2H), 7.07 (d, J=2.4 Hz, 1H), 3.88 (s, 3H).

### Synthesis of 4-(9-(6-Methoxynaphthalene-2-yl)-9H-fluorene-9-yl)phenol (Intermediate 2)

The entire amount of Intermediate 1 and 847 mg of phenol (9 mmol) were put into a 100 mL round bottom flask with a magnetic stirring bar and dissolved in 10 mL of dichloromethane. 20 drops of methane sulfonic acid were added dropwise thereto while stirring the mixture at room temperature. (A color of the reactant was changed into deep blue.)

After the mixture was stirred for about 18 hours under nitrogen atmosphere, a reaction end point was confirmed using thin layer chromatography (TLC). The resultant was cooled in an ice bath, and the reaction was terminated using 30 mL of saturated aqueous sodium bicarbonate solution. After the resultant was extracted with dichloromethane, and an organic layer was washed with Brine, dried over magnesium sulfate, filtered, and concentrated, thereby obtaining a crude product as a foam. The crude product as a foam was purified using a silica gel column (developing solution: 20% ethyl acetate/hexane), thereby obtaining 1.34 g of a white foamy material. Thereafter, it was confirmed using nuclear magnetic resonance (NMR) that the obtained material was 4-(9-(6-methoxynaphthalene-2-yl)-9H-fluorene-9-yl)phenol. Yield: 65%, ¹H NMR (700 MHz, CDCl₃) δ7.80 (d, J=7.5 Hz, 2H), 7.64 (d, J=8.7 Hz, 1H), 7.56-7.53 (m, 1H), 7.52 (d, J=1.8 Hz, 1H), 7.46 (d, J=7.6 Hz, 2H), 7.40-7.38 (m, 1H), 7.38-7.36 (m, 2H), 7.28 (td, J=7.5, 1.1 Hz, 2H), 7.14-7.11 (m, 2H), 7.10-7.07 (m, 2H), 6.72-6.69 (m, 2H), 3.90-3.88 (m, 3H).

### Synthesis of 4-hydroxyphenyl fluorene naphthol (Chemical Formula 1)

g of Intermediate 2 (2.44 mmol) was put into a 100 mL round bottom flask with a magnetic stirring bar and dissolved in 10 mL of dichloroethane. 5 mL (5 mmol) of boron tribromide solution (1.0M in hexane) was put thereinto at room temperature while maintaining nitrogen atmosphere, followed by heat at 60 degrees for 1 hour. A reaction end point was confirmed using thin layer chromatography (TLC). The resultant was cooled in an ice bath, and the reaction was terminated using 30 mL of saturated aqueous sodium bicarbonate solution. After the resultant was extracted with dichloromethane, and an organic layer was washed with Brine, dried over magnesium sulfate, filtered, and concentrated, thereby obtaining a yellow crude product as a foam. The crude product as a foam was purified using a silica gel column, and a ratio condition of developing solutions used at the time of purification was as follows.

(* Explanatory note for ratio sequence—ethyl acetate:hexane:dichloromethane)

1:8:1 (upper impurities separation)→

1:8:1+1 v/v % (intermediate impurities separation)→

1:4:1+1 v/v % methanol addition (target material separation)

As a result, 569 mg of a yellow foamy material having a purity of 99.2% was obtained (yield 58%). Thereafter, it was confirmed using NMR that the obtained material was 4-hydroxyphenyl fluorene naphthol of Chemical Formula 1. ¹H NMR (500 MHz, CDCl₃) δ7.78 (d, J=7.6 Hz, 2H), 7.57-7.51 (m, 2H), 7.48 (d, J=1.7 Hz, 1H), 7.43 (d, J=7.6 Hz, 2H), 7.39-7.33 (m, 3H), 7.28 (dd, J=7.5, 1.0 Hz, 2H), 7.26 (d, J=1.0 Hz, 1H), 7.12-7.09 (m, 2H), 7.07 (d, J=2.5 Hz, 1H), 7.00 (dd, J=8.8, 2.5 Hz, 1H), 6.72-6.67 (m, 2H), 5.07 (s, 1H), 4.81 (s, 1H).

### Synthesis of 1-benzylnaphthalene-2-ol (Intermediate 3)

1.44 g of naphthalene-2-ol, 1.23 mL of benzaldehyde, 50 mg of p-toluene sulfonic acid (catalyst), and 0.99 mL of pyrrolidine were all mixed with each other and put into a 35 mL sealed-microwave tube with a magnetic stirring bar, sealed, and irradiated with microwave at 900 W for 1 minute while stirring the mixture. However, in order to prevent over-heating, the irradiation was conducted two times for each 30 seconds.

The reactant was cooled to and maintained at room temperature, and 20 mL of methanol was added thereto and heated so that the resultant was entirely dissolved, followed by cooling, thereby obtaining a crystal. The crystal was filtered, washed with 5 mL of cold methanol two times, and then dried, thereby obtaining crystalline crude (yield: about 80%).

The obtained crystal was put into a 100 mL reactor with a reflux condenser, and 631 mg of ammonium formate and 15 mL of methanol were injected thereto, thereby dissolving the crystal. 60 mg of Pd/C, which was a reaction catalyst, was put thereinto, and a solvent was refluxed while maintaining a reaction temperature at 67° C. for 2 hours. A reaction end point was confirmed using thin layer chromatography (TLC), and the reactor was cooled and maintained at room temperature. The mixture was filtered using a celite bed, washed with methanol, and then concentrated. The obtained crude was dissolved in ethyl acetate and washed with water. An ethyl acetate layer was dried over sodium sulfate, filtered, and concentrated, thereby obtaining a solid. The obtained solid was washed with 4 mL of cold dichloromethane, thereby obtaining a solid (yield: 72%). Thereafter, it was confirmed using NMR that the obtained solid was 1-benzylnaphthalene-2-ol. ¹H NMR (300 MHz, CDCl₃) δ4.49 (2H, s), 7.07-7.23 (5H, m), 7.29-7.51 (2H, m), 7.76 (1H, d), 7.82 (1H, d), 7.93-7.98 (2H, m)

### Synthesis of di-substituted benzyl fluorene bisnaphthol ethanol (Chemical Formula 3)

70 ml of acetonitrile was put into a 250 mL reactor with a reflux condenser as a reaction solvent, 9-fluorenone, Intermediate 1, and 3-mercaptopropionic acid were sequentially injected thereinto at 25±2° C. and dissolved therein. A reaction temperature was maintained at 80° C. for 1 hour while slowly adding dropwise sulfuric acid, which is a reaction catalyst, and then, a reaction end point was confirmed using TLC.

The reactor was cooled and then, maintained at room temperature, and an aqueous potassium carbonate solution was added dropwise to neutralize the reaction solution, thereby obtaining crystalline crude. The obtained crude was recrystallized using hexane and dichloromethane, thereby obtaining di-substituted benzyl fluorene bisnaphthol intermediate having a high performance liquid chromatography (HPLC) purity of 99.1% (yield: 81%).

After 10 g of the obtained di-substituted benzyl fluorene bisnaphthol was put into a 500 mL 3-neck reactor with a reflux condenser and dissolved in dimethylsulfoxide, which was a reaction solvent, ethylene carbonate and 2-methylimidazole were sequentially put thereinto. A reaction end point was confirmed while refluxing the mixture at 145° C. After the reaction was terminate, the reactor was cooled to 50° C., and 100 mL of methanol was added dropwise to recrystallize the mixture, followed by cooling the reactor to room temperature. The obtained crude was dissolved in ethyl acetate and precipitated in distilled water to obtain di-substituted benzyl fluorene bisnaphthol ethanol of Chemical Formula 3, followed by drying under reduced pressure. Thereafter, it was confirmed using NMR that the obtained material was di-substituted benzyl fluorene bisnaphthol ethanol of Chemical Formula 3. Purity: 99.0%, yield: 45%, ¹H NMR (300 MHz, CDCl₃) δ3.87 (4H, t), 4.11 (4H, t), 4.49 (4H, s), 7.07-7.23 (10H, m), 7.29-7.51 (6H, m), 7.67-7.71 (4H, m), 7.75 (2H, d), 7.82 (2H, d), 7.93-7.98 (4H, m)

### Synthesis of 6-Hydroxy-2-naphthalenyl fluorene Derivative Based Homopolymer

A 4-hydroxyphenyl fluorene naphthol monomer having a purity of 99% or more was dissolved in a mixed solution of aqueous sodium hydroxide solution and dichloromethane, thereby obtaining a polymer through a carbonation reaction using phosgene gas. After Di-substituted benzyl fluorene bisnaphthol and di-substituted benzyl fluorene bisnaphthol ethanol were also prepared by the same synthesis method, gel permeation chromatography (GPC) molecular weights and glass transition temperatures (Tg) thereof were measured, and the results were shown in the following Table 1.

TABLE 1

| | GPC Molecular Weight | | | DSC | TGA |
|---|---|---|---|---|---|
| | Mn, | Mw, | | Tg | Td 5 wt |
| Sample | *1000 | *1000 | Mw/Mn | (° C.) | %(° C.) |
| 4-Hydroxyphenyl Fluorene | 15.7 | 56.9 | 3.62 | 139 | 378 |
| Naphthol | | | | | |
| Di-Substituted Benzyl | 16.2 | 60.1 | 3.71 | 148 | 391 |
| Fluorene Bisnaphthol | | | | | |
| Di-Substituted Benzyl | 13.1 | 32.4 | 2.47 | 147 | 399 |
| Fluorene Bisnaphthol | | | | | |
| Ethanol | | | | | |

Evaluation of Lens Properties

The polymer obtained during the process of synthesizing the di-substituted benzyl fluorene bisnaphthol monomer and a highly refractive resin reference (EP-5000, Mitsubishi Gas Chemical) were put into a mold of which a length and width is 2 cm and a thickness is 1 mm, respectively, and heated to thereby be melted. Then, plate type samples for evaluating optical properties of lens were manufactured by removing the mold, and refractive indexes, Abbe's numbers, and transmittance thereof were measured. The results were shown in the following Table 2.

TABLE 2

| | Refractive | | |
|---|---|---|---|
| | Index | ABBE | Transmit- |
| Sample | (587 nm, 25° C.) | number | tance |
| 4-Hydroxyphenyl Fluorene | 1.660 | 25 | 92% |
| Naphthol Based Polymer | | | |
| Di-Substituted Benzyl | 1.658 | 24 | 91% |
| Fluorene Bisnaphthol | | | |
| Based Polymer | | | |
| Di-Substituted Benzyl | 1.655 | 24 | 91% |
| Fluorene Bisnaphthol | | | |
| Ethanol Based Polymer | | | |
| Reference | 1.635 | 24 | 85% |

Referring to Tables 1 and 2, it may be appreciated that the polymer for a lens according to the present disclosure had a high refractive index of 1.655 or so, high transmittance of 90% or more, and a low glass transition temperature (Tg) suitable for injection ability.

As described above, in the 6-hydroxy-2-naphthalenyl fluorene derivative according to an exemplary embodiment of the present disclosure, the aryl group having a low molecular volume is introduced in one or more aryl moieties of the 6-hydroxy-2-naphthalenyl fluorene moiety, such that the polarity of the 6-hydroxy-2-naphthalenyl fluorene derivative itself may be increased, thereby making it possible to improve the refractive index.

Further, the alkyl group having a rotatable single bond is disposed at the front end of the aryl group, such that the glass transition temperature (Tg) may be decreased, and accordingly, the viscosity may be decreased, thereby making it possible to improve workability and moldability.

Since a halogen substituent such as bromine (Br) or chlorine (Cl) causing a dioxine problem is not introduced in the 6-hydroxy-2-naphthalenyl fluorene moiety, such that the 6-hydroxy-2-naphthalenyl fluorene derivatives according to an exemplary embodiment of the present disclosure may be eco-friendly.

Further, in the 6-hydroxy-2-naphthalenyl fluorene derivative according to an exemplary embodiment of the present disclosure, a sulfur (S) or nitrogen (N) atom generally introduced in order to improve optical properties is not used in the 6-hydroxy-2-naphthalenyl fluorene moiety, such that transparency may be secured.

As set forth above, the 6-hydroxy-2-naphthalenyl fluorene derivatives according to the present disclosure may have a high polarity and a low molecular volume, and the lens manufactured by molding the copolymer of the 6-hydroxy-2-naphthalenyl fluorene derivatives may have excellent optical properties.

Although the embodiment of the present disclosure has been disclosed for illustrative purposes, it will be appreciated that the present disclosure are not limited thereto, and those skilled in the art will appreciate that various modifications, additions and substitutions are possible, without departing from the scope and spirit of the disclosure.

Accordingly, any and all modifications, variations or equivalent arrangements should be considered to be within the scope of the disclosure, and the detailed scope of the disclosure will be disclosed by the accompanying claims.

What is claimed is:

1. A 6-hydroxy-2-naphthalenyl fluorene derivative represented by the following Chemical Formula 1: 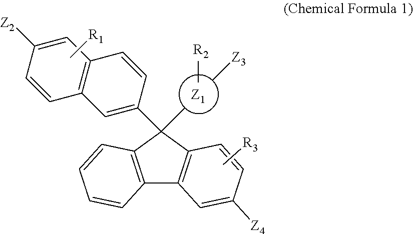
(In Chemical Formula 1, R₁, R₂, and R₃ are the same or different and are each hydrogen (H) or represented by the following Chemical Formula 2, at least one of R₁, R₂, and R₃ being selected from compounds represented by the following Chemical Formula 2; a substitution position of R₁ in a naphthyl group is not particularly limited; Z₁ is H or selected from a phenyl group, a naphthyl group, a biphenyl group, an anthryl group, and a phenanthryl group which have a C1-C4 alkyl substituent group; Z₂, Z₃, and Z₄ are the same or different and are each represented by an —H, —O—H, or —OCH₂CH₂O—H group, at least one of Z₂, Z₃, and Z₄ being —H; and a substitution position of Z₄ in a fluorene benzene ring of Chemical Formula 1 is not particularly limited; and
—R₄—Ar— [Chemical Formula 2]
in Chemical Formula 2, R₄ is a C1-C5 alkyl group and Ar is a C6-C22 aryl group).

2. The 6-hydroxy-2-naphthalenyl fluorene derivative according to claim 1, wherein Ar is selected from the group consisting of a phenyl group, a naphthyl group, a biphenyl group, an anthryl group, and a phenanthryl group.

3. The 6-hydroxy-2-naphthalenyl fluorene derivative according to claim 1, wherein the 6-hydroxy-2-naphthalenyl fluorene derivative represented by Chemical Formula 1 is a compound represented by the following Chemical Formula 3: 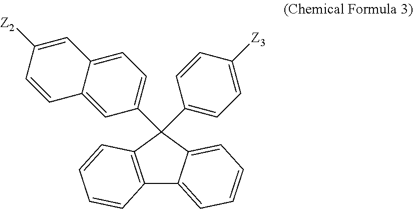
Z2 and Z3 are the same or different and are represented by an —O—H or —OCH₂CH₂O—H group.

4. The 6-hydroxy-2-naphthalenyl fluorene derivative according to claim 1, wherein the 6-hydroxy-2-naphthalenyl fluorene derivative represented by Chemical Formula 1 is a compound represented by the following Chemical Formula 4:

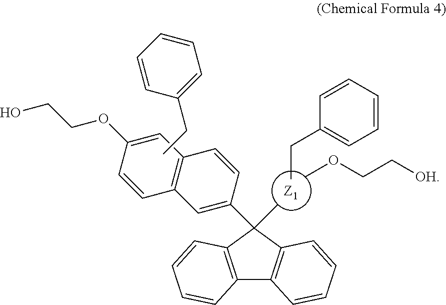

5. The 6-hydroxy-2-naphthalenyl fluorene derivative according to claim 1, wherein the 6-hydroxy-2-naphthalenyl fluorene derivative represented by Chemical Formula 1 is a compound represented by the following Chemical Formula 5:

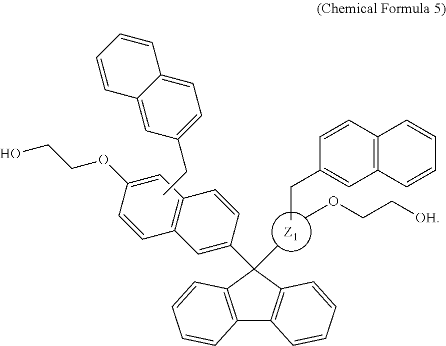

6. The 6-hydroxy-2-naphthalenyl fluorene derivative according to claim 1, wherein the 6-hydroxy-2-naphthalenyl fluorene derivative represented by Chemical Formula 1 is a compound represented by the following Chemical Formula 6:

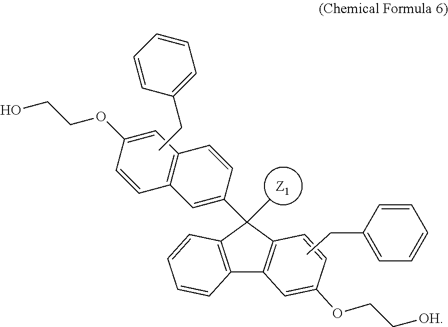

7. The 6-hydroxy-2-naphthalenyl fluorene derivative according to claim 1, wherein the 6-hydroxy-2-naphthalenyl fluorene derivative represented by Chemical Formula 1 is a compound represented by the following Chemical Formula 7:

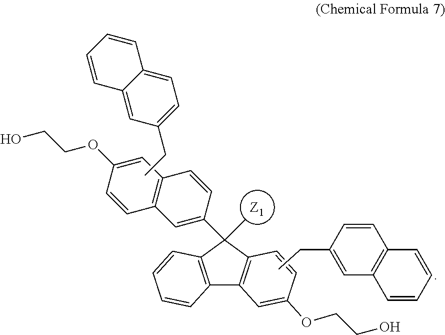

8. A copolymer of a compound represented by Chemical Formula 1: 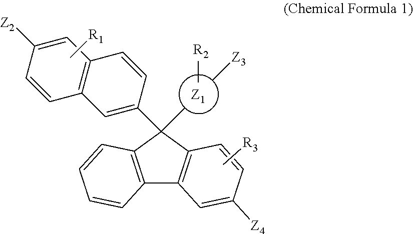
(In Chemical Formula 1, R₁, R₂, and R₃ are the same or different and are each hydrogen (H) or represented by the following Chemical Formula 2, at least one of R₁, R₂, and R₃ being selected from compounds represented by the following Chemical Formula 2; a substitution position of R₁ in a naphthyl group is not particularly limited, Z₁ is H or selected from a phenyl group, a naphthyl group, a biphenyl group, an anthryl group, and a phenanthryl group which have a C1-C4 alkyl substituent group; Z₂, Z₃, and Z₄ are the same or different and are each represented by an —H, —O—H, or —OCH₂CH₂O—H group, at least one of Z₂, Z₃, and Z₄ being —H; and a substitution position of Z₄ in a fluorene benzene ring of Chemical Formula 1 is not particularly limited; and
—R₄—Ar— [Chemical Formula 2]
in Chemical Formula 2, R₄ is a C1-C5 alkyl group and Ar is a C6-C22 aryl group).

9. A lens comprising a copolymer of a compound represented by the following Chemical Formula 1: 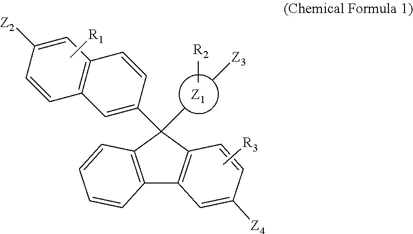
(In Chemical Formula 1, R₁, R₂, and R₃ are the same or different and are each hydrogen (H) or represented by the following Chemical Formula 2, at least one of R₁, R₂, and R₃ being selected from compounds represented by the following Chemical Formula 2; a substitution position of R₁ in a naphthyl group is not particularly limited, Z₁ is H or selected from a phenyl group, a naphthyl group, a biphenyl group, an anthryl group, and a phenanthryl group which have a C1-C4 alkyl substituent group; Z₂, Z₃, and Z₄ are the same or different and are each represented by an —H, —O—H, or —OCH₂CH₂O—H group, at least one of Z₂, Z₃, and Z₄ being —H; and a substitution position of Z₄ in a fluorene benzene ring of Chemical Formula 1 is not particularly limited; and
—R₄—Ar— [Chemical Formula 2]
in Chemical Formula 2, R₄ is a C1-C5 alkyl group and Ar is a C6-C22 aryl group).

10. A camera module comprising a lens containing a copolymer of a compound represented by the following Chemical Formula 1: 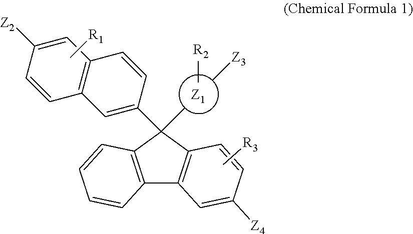
(In Chemical Formula 1, R₁, R₂, and R₃ are the same or different and are each hydrogen (H) or represented by the following Chemical Formula 2, at least one of R₁, R₂, and R₃ being selected from compounds represented by the following Chemical Formula 2; a substitution position of R₁ in a naphthyl group is not particularly limited, Z₁ is H or selected from a phenyl group, a naphthyl group, a biphenyl group, an anthryl group, and a phenanthryl group which have a C1-C4 alkyl substituent group; Z₂, Z₃, and Z₄ are the same or different and are each represented by an —H, —O—H, or —OCH₂CH₂O—H group, at least one of Z₂, Z₃, and Z₄ being —H; and a substitution position of Z₄ in a fluorene benzene ring of Chemical Formula 1 is not particularly limited; and
—R₄—Ar— [Chemical Formula 2]
in Chemical Formula 2, R₄ is a C1-C5 alkyl group and Ar is a C6-C22 aryl group).
